(19) United States
(12) Patent Application Publication
(10) Pub. No.: US 2026/0259171 A1
(43) Pub. Date: Sep. 3, 2026

(54) GAS SENSOR FET, GAS SENSOR, AND METHOD FOR MANUFACTURING GAS SENSOR FET

(71) Applicant: Hitachi High-Tech Corporation, Minato-ku, Tokyo (JP)

(72) Inventors: Hiroyuki UCHIYAMA, Tokyo (JP); Yoshitaka SASAGO, Tokyo (JP); Yuan BU, Tokyo (JP)

(21) Appl. No.: 19/163,239

(22) PCT Filed: Feb. 29, 2024
(86) PCT No.: PCT/JP2024/007569

§ 371 Date: Sep. 8, 2025

(30) Foreign Application Priority Data
Sep. 14, 2023 (JP) 2023-149041

(51) Int. Cl. G01N 27/414; G01N 33/00; H10D 64/27; H10D 64/66; H10P 95/70

(52) U.S. Cl. CPC G01N 27/4148; G01N 27/4141; G01N 33/0027; H10D 64/517; H10D 64/667; H10P 95/70

(57) ABSTRACT

A gas sensor FET with improved gas detection sensitivity is provided. The gas sensor FET includes a semiconductor substrate and a gate electrode layer formed on the semiconductor substrate and functioning as a gas detection unit. The gate electrode layer has a catalytic metal layer including a side exposed to an atmosphere. A side exposed to an atmosphere of the catalytic metal layer has a structure in which a plurality of dendritic-shaped structures, each including a plurality of fine structure portions smaller and finer than a columnar portion formed on a surface of the columnar portion, are arranged two-dimensionally. The plurality of dendritic-shaped structures are electrically connected to each other.

### TECHNICAL FIELD

The present invention relates to a gas sensor FET, a gas sensor, and a method for manufacturing a gas sensor FET.

### BACKGROUND ART

In the related art, a gas sensor for detecting specific gas or impurity gas has been known. An FET-type gas sensor is known as one type of gas sensor. PTL 1 discloses an example of an FET-type gas sensor.

### CITATION LIST

### Patent Literature

PTL 1: JP2018-115888A

### SUMMARY OF INVENTION

### Technical Problem

A semiconductor element called a gas sensor FET is used in an FET-type gas sensor. The gas sensor FET is small in size and is suitable for miniaturization of a gas sensor.

However, the gas sensor FET tends to have low sensitivity for detecting gas, and there is still room for improvement in the gas detection sensitivity.

An object of the invention is to further increase gas detection sensitivity of a gas sensor FET.

### Solution to Problem

One of representative embodiments of the invention relates to a gas sensor FET including: a semiconductor substrate; and a gate electrode layer formed on the semiconductor substrate and functioning as a gas detection unit, in which the gate electrode layer has a catalytic metal layer including a side exposed to an atmosphere, the side of the catalytic metal layer exposed to the atmosphere has a structure in which a plurality of dendritic-shaped structures, each including a plurality of fine structure portions smaller and finer than a columnar portion formed on a surface of the columnar portion, are two-dimensionally arranged, and the plurality of dendritic-shaped structures are electrically connected to each other.

In addition, one of representative embodiments of the invention relates to a gas sensor including: the gas sensor FET disposed on a substrate; a reference FET disposed on the substrate and configured to reference a gate threshold voltage; and a heater disposed on the substrate and configured to bring a temperature of the substrate closer to a predetermined temperature range.

In addition, one of representative embodiments of the invention relates to a method for manufacturing a gas sensor FET including a semiconductor substrate and a gas detection unit that is formed on the semiconductor substrate, constitutes a gate electrode layer, and includes a catalytic metal layer having a side exposed to an atmosphere, the method including: a catalytic metal layer forming step of forming the catalytic metal layer by sputtering at a sputtering pressure of 5 Pa or more.

### Advantageous Effects of Invention

According to embodiments of the invention, gas detection sensitivity of a gas sensor FET can be further increased.

### BRIEF DESCRIPTION OF DRAWINGS

FIG. 1 is a diagram showing an example of a structure of a gas sensor FET according to a first embodiment.

FIG. 2 is a diagram showing an example of a gate electrode layer of the gas sensor FET according to the first embodiment.

FIG. 3 is a flow diagram showing an example of a method for manufacturing the gas sensor FET according to the first embodiment.

FIG. 4 is a flow diagram showing an example of a method for forming the gate electrode layer according to the first embodiment.

FIG. 5 is a diagram illustrating that the gate electrode layer is formed after a protective layer is formed.

FIG. 6 is a diagram showing a relationship between a sputtering pressure during formation of a catalytic metal layer and specific surface area of the catalytic metal layer.

FIG. 7 is a diagram showing enlarged structural images of a plurality of Pt catalytic metal layers formed at different sputtering pressures.

FIG. 8 is a diagram showing a measurement example of a relationship between the very area of the catalytic metal layer and a variation amount of a gate threshold voltage with respect to a detection gas concentration in the gas sensor FET according to the first embodiment.

FIG. 9 is a diagram showing a measurement example of the variation amount of the gate threshold voltage with respect to a film thickness of the catalytic metal layer in the gas sensor FET according to the first embodiment.

FIG. 10 is a diagram showing a structure of a gate electrode layer of a gas sensor FET according to a second embodiment and a variation in a gate threshold voltage due to a change in a detection gas concentration in an atmosphere.

FIG. 11 is a diagram showing a structure of a gate electrode layer of a gas sensor FET according to a third embodiment and a variation in a gate threshold voltage due to a change in a detection gas concentration in an atmosphere.

FIG. 12 is a diagram showing a structure of a gate electrode layer of a gas sensor FET according to a fourth embodiment and a variation in a gate threshold voltage due to a change in a detection gas concentration in an atmosphere.

FIG. 13 is a diagram showing a structure of a gate electrode layer of a gas sensor FET according to a fifth embodiment and a variation in a gate threshold voltage due to a change in a detection gas concentration in an atmosphere.

FIG. 14 is a diagram illustrating a threshold voltage variation amount.

FIG. 15 is a diagram showing a relationship between a structure of the gate electrode layer and the gate threshold voltage variation amount.

### DESCRIPTION OF EMBODIMENTS

### History of Study Performed by Inventors

In order to realize a carbon-free society, a technology of using hydrogen to generate electricity, such as fuel cells, is attracting attention. As environmental awareness of people in the world is growing, there are expectations for an improvement in technology for fuels that do not discharge carbon dioxide. On the other hand, in recent years, in a case of a polymer electrolyte fuel cell (PEFC) used as a home fuel cell system and an automobile fuel cell, it is known that impurity gas such as carbon monoxide, hydrogen sulfide, and hydrocarbon compounds in high-concentration hydrogen gas causes deterioration in the cell, and it is said that a service life of the cell will be shortened unless a refresh operation is performed.

In addition, a hydrogen engine used in a distributed power source uses hydrogen gas containing impurities such as carburizing gas as fuel, and this also requires precise management of a combustion state after measuring an impurity concentration of the gas.

Therefore, technology for detecting impurities in high-concentration hydrogen is required. Gas sensor technology in the related art can measure the high-concentration hydrogen, but it is difficult to detect impurities contained in the high-concentration hydrogen.

Regarding this, it has been found that a semiconductor FET sensor formed from a stacked film of platinum (Pt) and titanium (Ti) (for example, as described in PTL 2: JP2009-300267A, and NPL 1: Applied Physics Express 3 (2010) 047201.) is capable of detecting impurities in high-concentration hydrogen to a certain extent. In a detection film of this sensor, a Ti layer introduced as an adhesive layer is oxidized to cause a structural change in an upper Pt layer, and many grain boundaries are formed. A structure in which many grain boundaries are formed in this way is referred to as a corridor structure. When a Pt layer having the corridor structure is formed on a gate electrode, a surface area of the Pt layer increases, which is considered to induce adsorption of hydrogen gas.

A specific surface area of these Pt corridor structures is said to be 0.1 m²/g to 10 m²/g, depending on a degree of oxidation of a base film. A detection mechanism of this sensor utilizes a matter that hydrogen gas adsorbed on a Pt catalyst layer is protonated, and the obtained protons are accumulated near a gate insulating layer, thereby generating an electric field, and a threshold voltage of an FET fluctuates. NPL 2 also describes a similar sensor in which island-shaped porous Pt formed from a solution is used for the detection film. A crystal grain size is described as about 0.5 μm, and it is considered that a specific surface area is almost the same as the above-described sensor having the corridor structure.

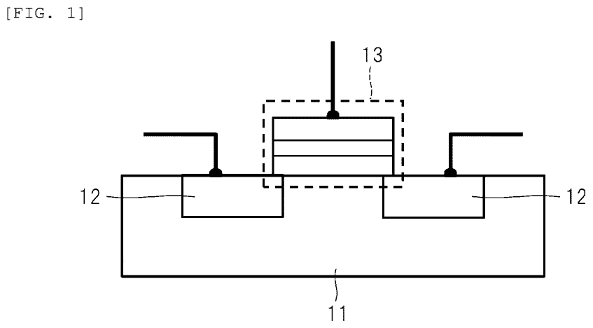

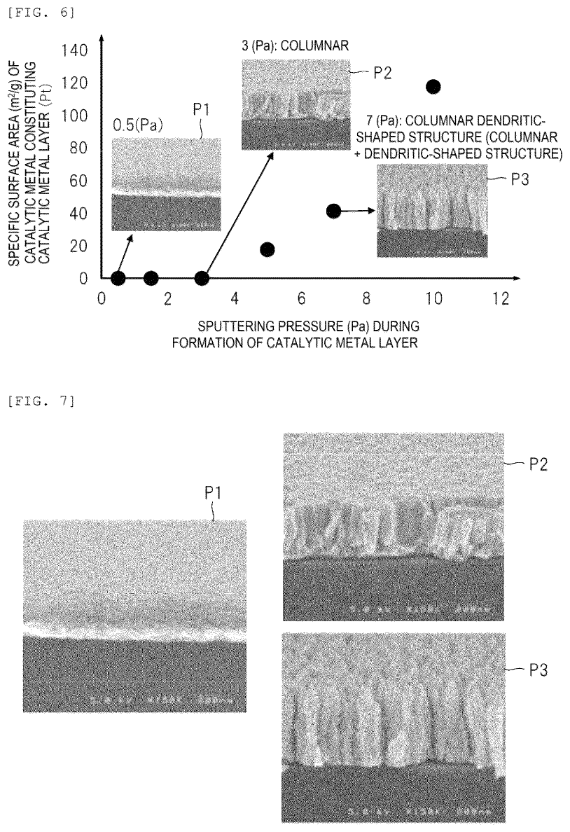

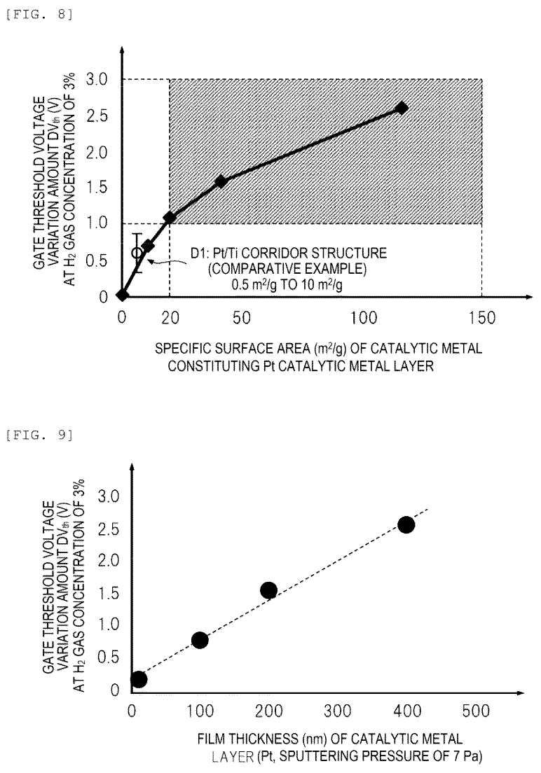

Other gas sensors for hydrogen detection and the like are as follows. PTL 3 (JP2012-013579A) describes technology for a chemical sensor using a sensitive film. Not limited to FET-type sensors, sensors that utilize properties such as changes in resistance or mass depending on a substance to which the sensitive film reacts can be produced by changing a type of sensitive film, making it possible to detect various chemical substances. The chemical sensor in FIG. 1 of PTL 3 is an example of a FET-type sensor, and FIGS. 7 and 8 of PTL 3 show examples of an FET-type sensor using a reference electrode.

As described in NPL 3 (Sensors and Actuators B 330 (2021) 129240), there are several detection principles for a hydrogen sensor which is one type of gas sensor. Among these sensors, an FET-type sensor, a capacitor-type sensor, and a diode-type sensor are classified as work-function-type sensors. The work-function-type sensor can be manufactured by a process using a semiconductor substrate, and thus, the work-function-type sensor is expected to have less cost, be more compact, and consume less power than sensors classified as other types.

As described in Table 2 of NPL 3, FET-type gas sensors are further classified into a plurality of types. Among these, a catalytic metal gate FET (referred to as a catalytic metal gate FET in NPL 2: Japanese Journal Of Applied Physics 37 (1998) 1100) has a simple structure similar to that of a normal MOSFET, and is therefore particularly suitable for miniaturization and cost reduction.

PTL 4 (JP2005-283578A) describes an FET-type gas sensor as one type of gas sensor. The FET-type gas sensor has a simple structure and is particularly suitable for miniaturization and cost reduction.

An FET sensor having a Pt/Ti corridor structure of PTL 3 is a mechanism that increases the number of gas adsorption sites to be sensed by utilizing a matter that a Pt layer, which is a catalytic metal, undergoes a structural change when a base metal is oxidized, and enables efficient detection, but similarly, a combination of the catalytic metal undergoing the structural change and an adhesive metal layer is limited, and in reality, there are very few options for effective catalytic metal layers.

Even if the grain boundary is effectively formed, the specific surface area is limited to about 10 m²/g. Furthermore, the specific surface area fluctuates depending on the degree of oxidation of the adhesive metal layer during manufacturing, and thus, there are concerns present in stable manufacturing. Therefore, it is impossible to select a catalytic metal layer that is suitable for the detection gas or a highly sensitive catalytic metal layer.

Under such a background, the inventors have invented a gas sensor FET having higher gas detection sensitivity as a result of intensive research. One of effects of embodiments of the invention is high sensitivity of gas detection in the gas sensor FET, and other effects include a wider range of options for a detection metal layer, a wider dynamic range, stability in manufacturing, and low cost. Hereinafter, embodiments of the invention will be described.

### Embodiment 1

### First Embodiment

FIG. 1 is a diagram showing an example of a structure of a gas sensor FET according to a first embodiment. As shown in FIG. 1, on a p-type semiconductor substrate 11 made of silicon or the like of a gas sensor FET 1, two n-type semiconductors 12, which constitute a drain and a source, are formed at an interval from each other, and a gate electrode layer 13 is formed to cover a part of each of the two n-type semiconductors 12. When gas is adsorbed by the gate electrode layer 13, a threshold voltage of the FET changes. By measuring the change in the threshold voltage, the gas can be detected. In other words, the gate electrode layer 13 functions as a gas detection unit.

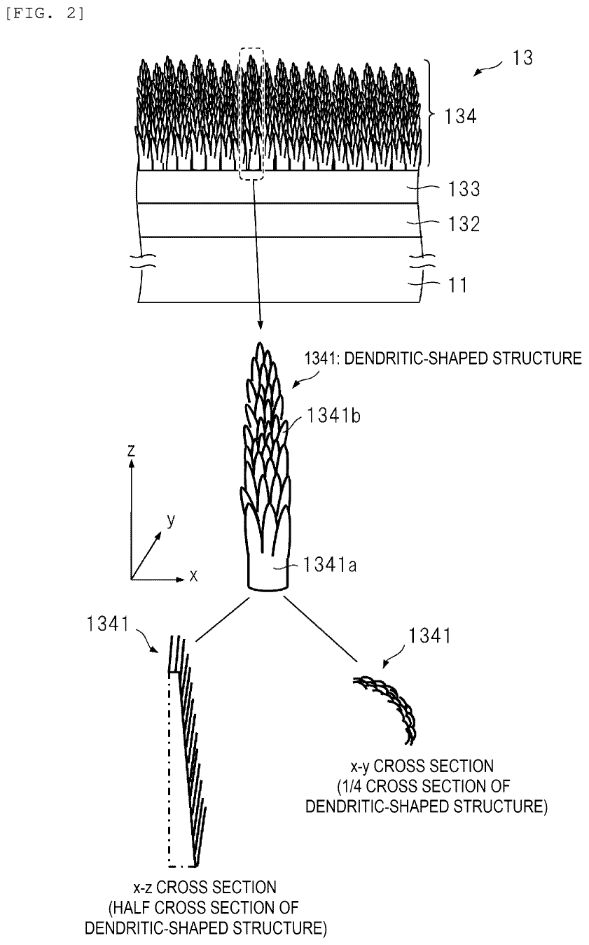

FIG. 2 is a diagram showing an example of a gate electrode layer of the gas sensor FET according to the first embodiment. In the gate electrode layer 13 shown in FIG. 2, a gate insulating layer 132 is formed on the semiconductor substrate 11, an oxide layer (for example, a TiO₂ film having a thickness of 10 nm) 133 is formed on the gate insulating layer 132, and a catalytic metal layer (for example, a Pt layer having a specific surface area of about 40 m²/g and a thickness of 20 nm) 134 having a characteristic structure of this embodiment is formed on the oxide layer 133. The oxide layer 133 corresponds to an adhesive layer in a gate electrode layer having a corridor structure in the related art. That is, the gate electrode layer 13 has the oxide layer 133 in contact with the catalytic metal layer 134 and disposed on a side opposite to a side exposed to an atmosphere.

As shown in upper and middle parts of FIG. 2, the characteristic structure of the gate electrode layer 13 according to the first embodiment is a columnar dendritic-shaped structure in which a plurality of dendritic-shaped structures 1341, each including a plurality of fine structure portions 1341b smaller and finer than a columnar portion 1341a formed on a surface of the columnar portion 1341a, are arranged two-dimensionally. The plurality of dendritic-shaped structures 1341 are electrically connected to each other. Here, a column axis direction of the columnar portion 1341a is a z-axis direction, one direction in a plane perpendicular to the z-axis direction is an x-axis direction, and a direction perpendicular to the x-axis direction in the same perpendicular plane is a y-axis direction. A lower part of FIG. 2 shows an x-z cross section (half cross section of the dendritic-shaped structure) of the dendritic-shaped structure 1341 and an x-y cross section (1/4 cross section of the dendritic-shaped structure) of the dendritic-shaped structure 1341. As can be seen from these figures, in the dendritic-shaped structure 1341, a large number of thin plate-shaped, leaf-shaped, or grain-shaped fine structure portions 1341b, which resemble the thin leaves of a tree or petals and extend to include a component of a direction where a film grows during film formation in the z-axis direction, are formed to almost completely cover the surface of the columnar portion 1341a.

According to the shape of the dendritic-shaped structure 1341, a specific surface area of a catalytic metal corresponding to a gas sensor in the gate electrode layer 13 is increased compared to that of the corridor structure in the related art, and gas detection sensitivity is improved. Hereinafter, a method for manufacturing a gas sensor FET having a catalytic metal film of such a dendritic-shaped structure in the gate electrode layer 13 will be described.

### Method for Manufacturing Gas Sensor FET

The method for manufacturing a gas sensor FET according to the present embodiment has a characteristic in a method for forming a gate electrode layer, and is not particularly different from a method for manufacturing a general MOSFET, except for the gate electrode. Therefore, here, an outline of steps of manufacturing the gas sensor FET according to the present embodiment is described first, and then the method for forming the gate electrode layer of the gas sensor FET of the present embodiment is mainly described.

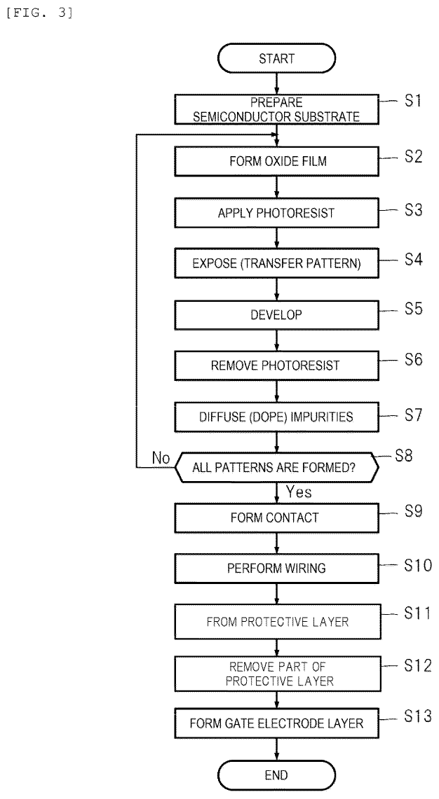

FIG. 3 is a flow diagram showing an example of the method for manufacturing the gas sensor FET according to the first embodiment. This flow diagram includes only main processes, and detailed processes are omitted.

As shown in FIG. 3, first, in step S1, a step of preparing a semiconductor substrate is performed. That is, a step of cutting a semiconductor substrate (also referred to as a wafer or silicon wafer) from a silicon ingot and polishing the cut semiconductor substrate is performed.

Next, in step S2, a step of forming an oxide film is performed. That is, a step of forming an insulating oxide film necessary for forming, on a surface of the semiconductor substrate, an insulating layer between wiring layers of an FET is performed.

Next, in step S3, a step of applying a photoresist is performed. That is, a step of applying a photoresist onto the oxide film formed on the surface of the semiconductor substrate is performed.

Next, in step S4, an exposure step is performed. That is, a step of covering the semiconductor substrate with a mask, irradiating the semiconductor substrate with light such as ultraviolet light, and transferring a necessary pattern onto the oxide film is performed.

Next, in step S5, a development step is performed. That is, a step of developing the photoresist, leaving the photoresist on the oxide film onto which the pattern is transferred, and removing other photoresists is performed.

Next, in step S6, a step of removing an etching photoresist is performed. That is, a step of removing the photoresist, and at the same time, removing unnecessary portions of the oxide film where the photoresist is removed in step S5.

Next, in step S7, a step of diffusing (doping) impurities is performed. That is, a step of diffusing impurities by passing impurity gas through the surface of the semiconductor substrate is performed.

Next, in step S8, a step of determining whether all necessary patterns are formed is performed. Note that formation of the gate electrode layer is not included in the formation of the necessary pattern. This is because the gate electrode layer needs to be exposed to the atmosphere so that the gate electrode layer can function as a gas sensor, and thus, as described below, after forming a protective layer, a part of the protective layer needs to be removed, and the gate electrode layer needs to be formed in a portion having no protective layer as a final step.

If it is determined in this determination that all necessary patterns are formed and a chip is completed except for the gate electrode layer part (S8: Yes), the process step proceeds to step S9. If it is determined that all necessary patterns are not formed (S8: No), the process step returns to step S2, and steps from forming the oxide film to performing the doping are performed again.

Next, in step S9, a step of forming contacts is performed. That is, a step of forming contacts is performed by performing a predetermined process on a necessary portion so that a surface of the chip is in a state where wiring is possible.

Next, in step S10, a wiring step is performed. That is, a step of performing wiring between contacts by connecting a wiring material or the like is performed. The wiring material used here is a material having oxidation resistance. As will be described later, it is necessary that after the wiring is performed, a protective layer is formed, a part of the protective layer is removed, and a gate insulating layer is formed there, and when this gate insulating layer is formed, a strong oxidation process is performed on the semiconductor substrate. This is to prevent the wiring material that is disposed earlier from being altered by oxidation in this case. Examples of the material having oxidation resistance include tungsten, which is resistant to oxidation, and metal materials with high-melting point or oxidation-resistant metal materials, such as LaB₆ and CuMn.

Next, in step S11, a step of forming the protective layer is performed. That is, in order to prevent the chip from being contaminated or deteriorated due to external gases, atmosphere, or the like, a step of covering the entire chip with the protective layer is performed.

Next, in step S12, a step of removing a part of the protective layer for forming the gate electrode layer is performed. For example, the protective layer covering a region where the gate electrode layer is to be formed is removed by dry etching or wet etching.

Next, in step S13, a step of forming the gate electrode layer is performed. For example, the gate insulating layer (having a film thickness of, for example, 15 nm) is formed on the semiconductor substrate, an oxide film is formed on the gate insulating layer, and a catalytic metal layer is further formed on the oxide film to form the gate electrode layer.

### Method for Forming Gate Electrode Layer

Here, an example of the method for forming the gate electrode layer according to the present embodiment will be described.

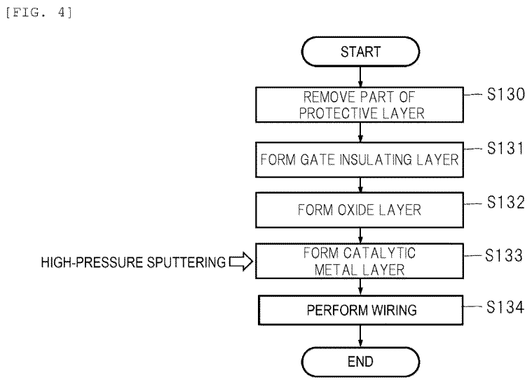

FIG. 4 is a flow diagram showing an example of the method for forming the gate electrode layer according to the first embodiment. FIG. 5 is a diagram illustrating that the gate electrode layer is formed after the protective layer is formed. As described above, a part of the gate electrode layer that functions as a detection film of a sensor, particularly the catalytic metal layer, is required to be exposed to the outside. Therefore, in order to form the gate electrode layer including the gate insulating layer, it is necessary to form the protective layer (passivation film), then provide an opening in the protective layer, and form the gate electrode layer in the opening (see FIG. 5).

First, as a premise, as shown in FIG. 5, it is assumed that a protective layer 14 is provided on the semiconductor substrate 11. As shown in FIG. 4, in step S130, a step of removing a part of the protective layer 14 provided on the semiconductor substrate 11 that corresponds to the region where the gate electrode layer is to be formed is performed.

Next, in step S131, a step of forming the gate insulating layer on the semiconductor substrate 11 is performed. specifically, the gate insulating layer 132 is formed at a portion of the semiconductor substrate 11 where the protective layer 14 is removed, by using a well-known method such as a sputtering method or a plasma CVD method. A film thickness of the gate insulating layer 132 is, for example, about 50 nm to 100 nm.

For example, the gate insulating layer 132 is formed by using tetra ethoxy silane (TEOS) or monosilane as raw materials under a condition of about 200° C. to 500° C. by the plasma CVD method or the like. In this case, since the exposure is performed under strong oxidation conditions, there is a risk that the wiring material formed beforehand, such as Al, may be oxidized. Therefore, in the gas sensor FET according to the present embodiment, the wiring material is preferably made of an oxidation-resistant metal material. As the oxidation-resistant metal material, for example, tungsten, lanthanum hexaboride (LaB₆), copper manganese (CuMn), or other high melting point metals or oxidation-resistant alloys are effective.

Next, in step S132, a step of forming an oxide layer is performed. The oxide layer 133 is an oxide layer corresponding to an adhesive layer in the gate electrode having the Pt/Ti corridor structure in the related art. In the present embodiment, the oxide layer—133 is not necessarily required, but can be used to adjust the detection gas and detection sensitivity by combining with the catalytic metal layer 134. For example, a TiO₂ film, a YSZ film, or a GDC layer is formed as the oxide layer 133. For example, a sputtering method is used for the film formation. For example, a magnetron sputtering system is used to perform sputter deposition using a target having a diameter of 2 inches, such as Ti 02, YSZ (8% Y₂O₃, 92% ZrO₂), and GDC layer (10% Gd₂O₃, 90% CeO₂), under conditions of an Ar gas pressure of 0.5 Pa and a high-frequency discharge power of 50 W.

Next, in step S133, a step of forming a catalytic metal layer is performed. The catalytic metal layer 134 of Pt, Ni, or the like having a columnar dendritic-shaped structure according to the present embodiment is formed. A high-pressure sputtering method is used for this film formation. For example, a magnetron sputtering system is used to perform sputter deposition using a Pt target having a diameter of 2 inches under a condition of an Ar gas pressure of 7.0 Pa, a direct current discharge power of 50 W, or a radio-frequency discharge power of 50 W. By performing sputtering under the high-pressure condition, the catalytic metal layer 134 having a dendritic-shaped structure and a specific surface area of 40 m²/g or more is formed. For example, a Pt layer having a thickness of 25 nm is formed as the catalytic metal layer—134.

Here, an example of the Pt catalytic metal layer having a thickness of 25 nm has been described, but when comparing with the Pt catalytic metal layer (having a film thickness of, for example, 15 nm) having the Pt/Ti corridor structure in the related art, good gate threshold voltage variations are confirmed in hydrogen detection in an atmosphere with 3% hydrogen even if about half the amount of Pt is used.

Furthermore, when the lower oxide layer is made of a yttria stabilized zirconia (YSZ) film (having a film thickness of, for example, 10 nm) which is a solid electrolyte film, the gate threshold voltage variation settles quickly, and good detection characteristics are shown. Here, the YSZ film is formed by a magnetron sputtering system using a Y₂O₃ (8 mol %)-ZrO₂ (92 mol %) target having a diameter of 4 inches under a radio-frequency discharge power of 200 W. This is thought to be because hydrogen gas is adsorbed by the Pt catalytic metal layer, becomes protons, and followed by being quickly transported to a gate insulating layer side.

Next, in step S134, a wiring step is performed. That is, a step of connecting the gate electrode layer 13 and a contact with a wiring material is performed.

By performing the above steps, the gas sensor FET 1 according to the present embodiment is completed. The gate electrode layer 13 functioning as the gas detection unit can be configured to be exposed to the atmosphere without being blocked by the protective layer 14.

Note that, the method for manufacturing a gas sensor FET includes a surface treatment step of activating a catalytic effect by performing surface treatment of oxidation or reduction on the formed catalytic metal layer after the step of forming the catalytic metal layer.

Furthermore, after the above-described catalytic metal layer 134 is formed, insulating layers such as Si₂N₃/SiO₂ or AlN/Al₂O₃ are formed with film thicknesses of, for example, 100 nm or more by sputtering or the like, so that a reference FET in which the gate electrode layer 13 has no externally exposed portion can be manufactured.

### Relationship between Sputtering Pressure During Formation of Catalytic Metal Layer and Specific Surface Area of Catalytic Metal Layer

Hereinafter, a relationship between a sputtering pressure during formation of the catalytic metal layer and the specific surface area of the catalytic metal layer—will be described.

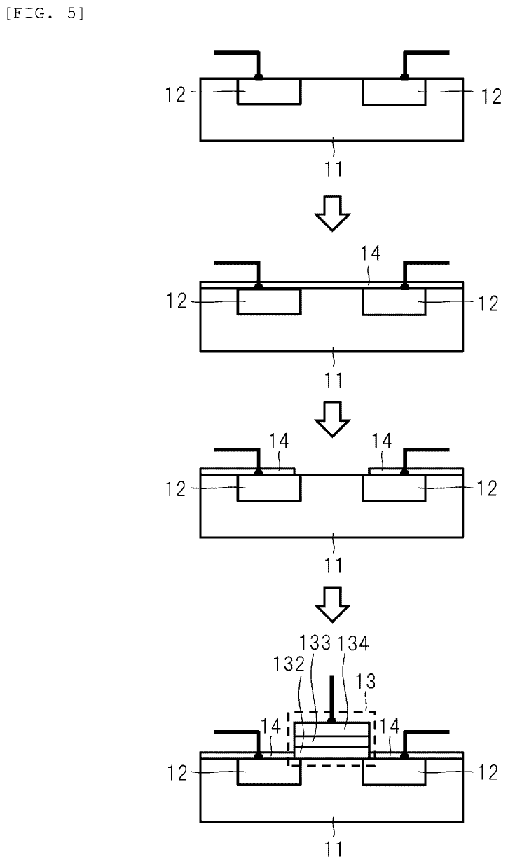

FIG. 6 is a diagram showing the relationship between the sputtering pressure during the formation of the catalytic metal layer and the specific surface area of the catalytic metal layer. FIG. 7 is a diagram showing enlarged structural images of a plurality of Pt catalytic metal layers formed at different sputtering pressures.

As shown in FIG. 6, when the sputtering pressure during the formation of the catalytic metal layer is less than 2 Pa, the catalytic metal layer has a substantially flat surface as shown in an image P1. In this case, a specific surface area is low. When the sputtering pressure is 3 Pa, the catalytic metal layer has a columnar structure in which a plurality of columnar portions are arranged two-dimensionally as shown in an image P2, but the plurality of columnar portions with smooth surfaces are arranged almost without gaps, and thus, the specific surface area remains low. On the other hand, when the sputtering pressure is 5 Pa or more, as shown in an image P3, the catalytic metal layer has a columnar structure while a surface of the columnar portion becomes a dendritic-shaped structure, so that the catalytic metal layer has a columnar dendritic-shaped structure. Therefore, the specific surface area becomes large, and it is considered that the higher the sputtering pressure, the more the dendritic-shaped structures grow and the larger the specific surface area becomes.

### Measurement Example of Variation in Gate Threshold Voltage with respect to Specific Surface Area of Catalytic Metal Layer

Here, a measurement example of the variation in the gate threshold voltage with respect to the specific surface area of the catalytic metal layer will be described.

FIG. 8 is a diagram showing a measurement example of a relationship between the very area of the catalytic metal layer and a variation amount of a gate threshold voltage with respect to a detection gas concentration in the gas sensor FET according to the first embodiment. FIG. 8 shows the measurement example of the relationship between the specific surface area of the catalytic metal layer in the gas sensor FET according to the first embodiment and a variation amount DVₜₕ of a gate threshold voltage Vₜₕ in an atmosphere with a hydrogen gas concentration of 3% (hereinafter, also referred to as a gate threshold voltage variation amount DVₜₕ). In this measurement example, a detection film structure obtained by forming the Pt catalytic metal layer having the columnar dendritic-shaped structure on the gate insulating layer is adopted as the gate electrode layer of the gas sensor FET. The catalytic metal layer has a film thickness of 25 nm.

As shown in FIG. 8, when the specific surface area of the catalytic metal layer is 20 m²/g or more, the gate threshold voltage variation amount DVₜₕ becomes 1.0 V or more. When the specific surface area of the catalytic metal layer is 120 m²/g, the gate threshold voltage variation amount DVₜₕ exceeds 2.5 V. In a case of the Pt/Ti corridor structure in the related art, as shown by D1 of FIG. 8, the specific surface area of the catalytic metal layer is 0.5 m²/g to 10 m²/g, and the gate threshold voltage variation amount DVₜₕ is 0.4 V to 0.9 V. Compared with a case where the gate electrode layer has the Pt/Ti corridor structure in the related art (D1), in the present embodiment in which the gate electrode layer has the Pt catalytic metal layer having the columnar dendritic-shaped structure, the gate threshold voltage variation amount DVₜₕ can be expected to be 1.0 V or more and 3.0 V or less when the specific surface area of the catalytic metal layer is in a range of 20 m²/g or more and 150 m²/g or less, and it can be seen that a sufficiently practical gas detection sensitivity is obtained.

### Measurement Example of Variation in Gate Threshold Voltage with Respect to Film Thickness of Catalytic Metal Layer

Next, a measurement example of the variation in the gate threshold voltage with respect to the film thickness of the catalytic metal layer will be described.

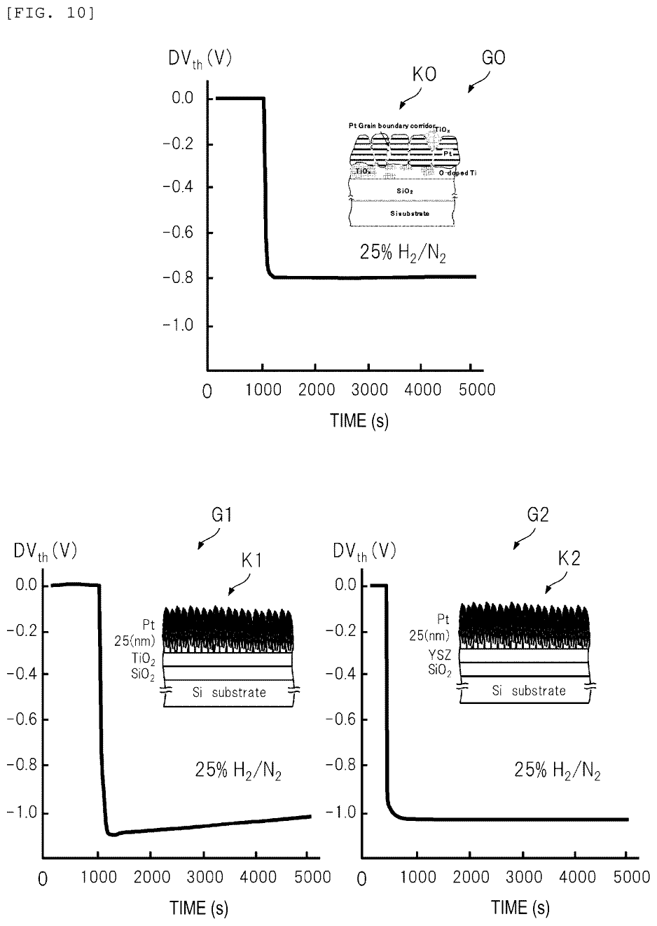

FIG. 9 is a diagram showing a measurement example of the variation amount of the gate threshold voltage with respect to the film thickness of the catalytic metal layer in the gas sensor FET according to the first embodiment. In this measurement example, a detection film structure obtained by forming the catalytic metal layer having the columnar dendritic-shaped structure according to the present embodiment on the gate insulating layer is adopted as the gate electrode layer of the gas sensor FET. The atmosphere is nitrogen gas with a hydrogen gas concentration of 3% (3% H₂/N₂). The catalytic metal layer is Pt, and is formed by sputtering at a sputtering pressure of 7 Pa.

As can be seen from FIG. 9, as the film thickness of the catalytic metal layer having the columnar dendritic-shaped structure increases, an area of adsorption sites in the gate electrode layer of the gas sensor FET increases, and the gate threshold voltage variation amount DVₜₕ becomes larger. In principle, the larger the film thickness of the catalytic metal layer, the larger the area of the gas adsorption sites, and the higher the sensitivity can be expected. However, in the catalytic metal layer having the columnar dendritic-shaped structure, deterioration of adhesion and film peeling easily occur due to an increase in an aspect ratio of the columnar dendritic-shaped structure. Therefore, according to the experience of the inventors, it is considered that the film thickness of the catalytic metal layer having the columnar dendritic-shaped structure is preferably up to about 500 nm.

In addition, regarding the manufacture of the gas sensor FET, in the case of the Pt/Ti corridor structure in the related art, there is structural variation due to an oxidation state of the Ti layer, whereas in the case of the columnar dendritic-shaped structure according to the present embodiment, the structural variation simply depends only on the film formation conditions. Therefore, according to the present embodiment, gas sensor FET can be manufactured stably and uniformly. Furthermore, according to the present embodiment, it is also possible to form a catalytic metal layer having the columnar dendritic-shaped structure directly on a gate insulating layer without introducing an adhesive layer under the catalytic metal layer, and to use the manufactured FET as a gas sensor.

### Second Embodiment

Next, a second embodiment will be described.

FIG. 10 is a diagram showing a structure of a gate electrode layer of a gas sensor FET according to the second embodiment and a variation in a gate threshold voltage Vth due to a change in a detection gas concentration in an atmosphere. As shown in structures K1 and K2 in FIG. 10, the gas sensor FET according to the second embodiment uses a Pt (25 mm thick) film having a columnar dendritic-shaped structure as a catalytic metal layer in the gate electrode layer, and uses silicon oxide (SiO₂) as the gate insulating layer. However, in the structure K1, an oxide layer corresponding to the adhesive layer is titanium oxide (TiO₂), and in the structure K2, an oxide layer is yttria stabilized zirconia (YSZ). The detection gas is hydrogen (H₂) in nitrogen (N₂).

For comparison, an upper part of FIG. 10 also shows a structure K0 of a gate electrode of the gas sensor FET in the related art and the variation of the gate threshold voltage Vₜₕ due to the change in the detection gas concentration in the atmosphere. The structure K0 is a Pt/Ti corridor structure, the adhesive layer is titanium oxide, and the gate insulating layer is silicon oxide.

Graphs G0 to G2 in FIG. 10 show the gate threshold voltage variation amount DVₜₕ when the atmosphere is initially 100% N₂ and then changed to 25% H₂ concentration. As can be seen from graphs G1 to G2, even if an amount of Pt used in the Pt catalytic metal layer is smaller than that of the corridor structure in the related art, a threshold voltage variation larger than that in the corridor structure in the related art is confirmed.

### Third Embodiment

Next, a third embodiment will be described.

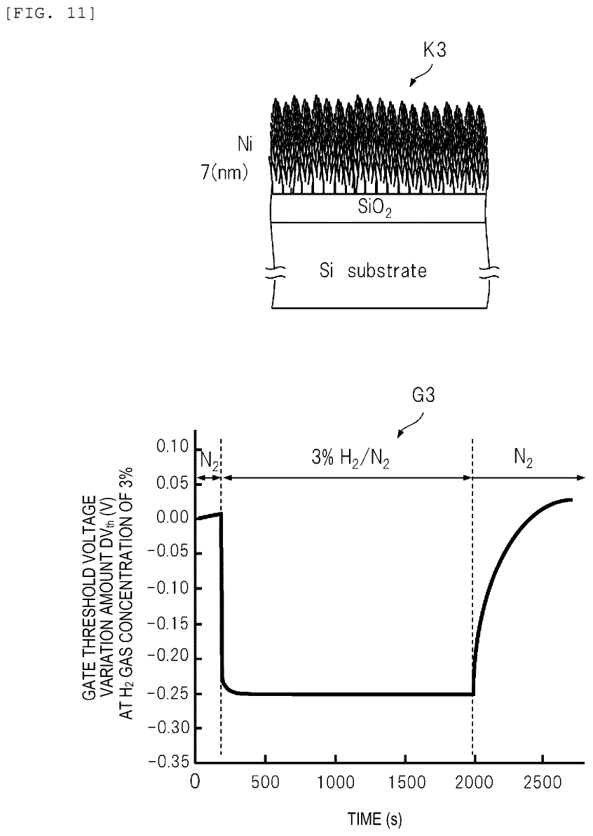

FIG. 11 is a diagram showing a structure of a gate electrode layer of a gas sensor FET according to the third embodiment and a variation in a gate threshold voltage Vₜₕ due to a change in a detection gas concentration in an atmosphere. As shown in a structure K3 of FIG. 11, the gas sensor FET according to the third embodiment uses Ni having a columnar dendritic-shaped structure as the catalytic metal layer of the gate electrode, and uses silicon oxide (SiO₂) as the gate insulating layer. An oxide layer corresponding to the adhesive layer is not provided. The detection gas is hydrogen (H₂) in nitrogen (N₂). Ni is oxidized in the air, making it difficult to perform catalytic action, but Ni can be used for detection in a reducing atmosphere such as hydrogen.

As for Ni having the columnar dendritic-shaped structure, similar to the first embodiment, a Ni target having a diameter of 2 inches is subjected to sputtering deposition under conditions of an Ar gas pressure of 7.0 Pa and a radio-frequency power of 50 W by a magnetron sputtering system. By performing sputtering under high pressure conditions, a Ni catalytic metal layer having a specific surface area of 40 m²/g or more and a columnar dendritic-shaped structure is formed.

Here, a 7 nm thick Ni having a columnar dendritic-shaped structure is used as the catalytic metal layer of the gate electrode of the gas sensor FET. Unlike the Pt/Ti corridor structure in the related art, a Ti layer or an adhesive layer corresponding to Ti oxide is not required, and the Ni layer having the columnar dendritic-shaped structure can be directly formed and used as the gate electrode. Since the Ni layer having the columnar dendritic-shaped structure is prone to natural oxidation, the Ni layer is subjected to a reduction treatment at 200° C. in a hydrogen atmosphere immediately before measurement, and then measured.

A graph G3 in FIG. 11 shows a variation of the gate threshold voltage Vth when the atmosphere is initially 100% N₂, changed to one containing 3% H₂ concentration midway, and then changed back to 100% N₂. As can be seen from the graph G3, the variation in the threshold voltage is confirmed to be similar to that of a Pt-based catalytic metal layer. It is difficult to form a catalytic metal layer such as Ni into a columnar dendritic-shaped structure in the manufacturing method in the related art, and a reason why this matter is possible is largely due to the method for forming a gate electrode layer by sputtering according to the present embodiment.

### Fourth Embodiment

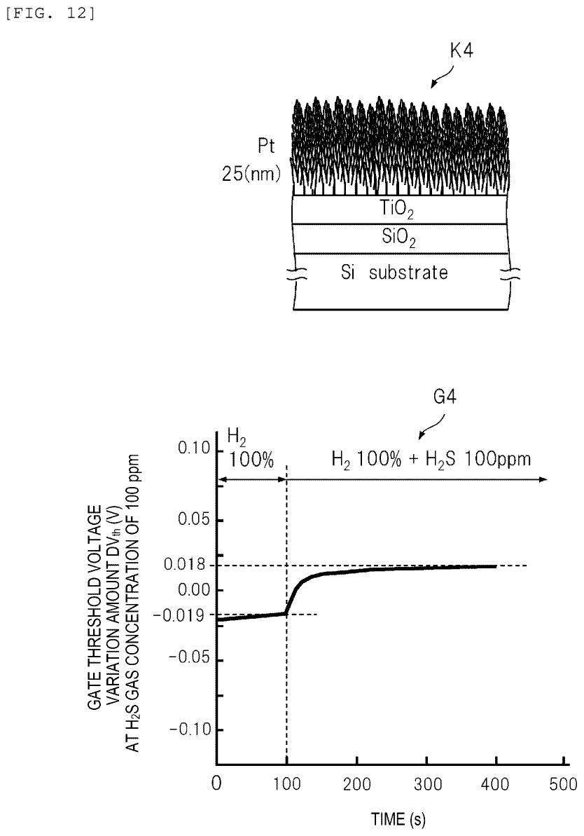

A fourth embodiment will be described. FIG. 12 is a diagram showing a structure of a gate electrode layer of a gas sensor FET according to the fourth embodiment and a variation in a gate threshold voltage Vth due to a change in a detection gas concentration in an atmosphere. The gas sensor FET according to the fourth embodiment uses, as a gate electrode, one in which a Pt catalytic metal layer having a columnar dendritic-shaped structure is laminated on a titanium oxide film, and the detection gas is hydrogen sulfide (H₂S). A film thickness of the Pt catalytic metal layer having the columnar dendritic-shaped structure is 25 nm.

A graph G4 in FIG. 12 shows the variation in the gate threshold voltage Vₜₕ when the gas sensor FET according to the fourth embodiment is placed in an atmosphere of 100% hydrogen and 100 ppm of hydrogen sulfide gas is introduced. From this graph G4, it can be seen that the gate threshold voltage fluctuates due to hydrogen sulfide, which is impurity gas, even in high-concentration hydrogen. Note that regarding an amount of a catalytic metal used for gas detection, compared with the gas sensor FET having a catalytic metal/oxide corridor structure in the related art, 50% or less of the using amount of Pt achieves substantially the same gate threshold voltage variation.

### Fifth Embodiment

A fifth embodiment will be described.

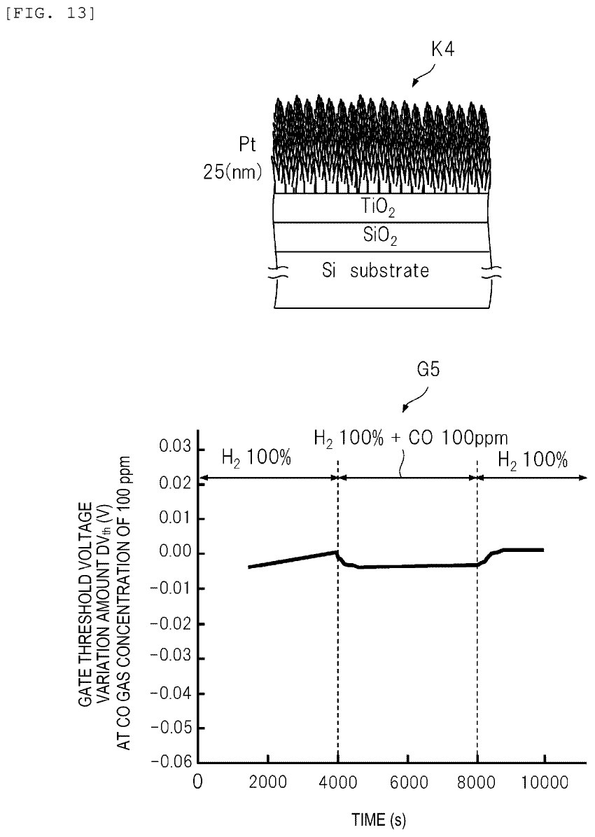

FIG. 13 is a diagram showing a structure of a gate electrode layer of a gas sensor FET according to the fifth embodiment and a variation in a gate threshold voltage Vₜₕ due to a change in a detection gas concentration in an atmosphere. A gate electrode structure of the gas sensor FET according to the fifth embodiment is a structure of K4, which is the same as that of the fourth embodiment, but the measurement of the variation in the gate threshold voltage Vth is performed under conditions different from those of the fourth embodiment. Specifically, the gas sensor FET same as that of the fourth embodiment is placed in an atmosphere of 100% hydrogen concentration, 100 ppm of carbon monoxide is introduced into the atmosphere, and the variation in the gate threshold voltage when the atmosphere is returned to 100% hydrogen concentration is measured.

As can be seen from a graph G5 in FIG. 13, the gate threshold voltage Vth fluctuates with the introduction of 100 ppm of carbon monoxide. The sensitivity of gas detection can be improved by increasing a thickness (film thickness) of a catalytic metal layer having a porous structure.

By utilizing the above-described effects of the gas sensor FET according to the fifth embodiment, it becomes possible to detect impurity gas in high-concentration hydrogen gas, which is a cause of deterioration of the catalyst in fuel cells. For example, calculations can be made based on an integrated value of the exposed impurity gas and reflected in a refresh operation of the PEFC, thereby contributing to a longer life of the fuel cell. In addition, regarding a hydrogen engine used in an automobile and a distributed power source, detection data of impurity gas in high-concentration hydrogen can be utilized in engine control in real time, which enables optimization of combustion efficiency and stable operation.

### Characteristics of Method for Manufacturing Gas Sensor FET According to Embodiment

Next, characteristics of a method for manufacturing a gas sensor FET according to the present embodiment will be described. The method for manufacturing a gas sensor FET according to the present embodiment is based on a method for manufacturing a general MOSFET, but differs significantly in the following three points.

A first difference is that the catalytic metal layer of the gate electrode is required to be made into a porous structure to increase the specific surface area of the catalytic metal layer, and thus, the catalytic metal layer is formed by sputtering.

A second difference is that the gate electrode layer portion that functions as a gas detection film, particularly the catalytic metal layer, is required to be exposed to the outside, and thus, after the protective layer is formed, the gate electrode layer is formed by removing a part of the protective layer.

A third difference is that the gate insulating layer, which is a part of the gate electrode layer, is formed after the protective layer is formed, and thus, the gate insulating layer is required to be formed after the wiring material is connected. Therefore, the wiring material is exposed to a strong oxidizing environment when the gate insulating layer is formed, and thus, the wiring material is made of an oxidation-resistant metal material.

### Relationship between Structure of Gate Electrode and Gate Threshold Voltage Variation Amount

Here, results of an investigation of a relationship between a structure of the gate electrode layer and the gate threshold voltage variation amount are described.

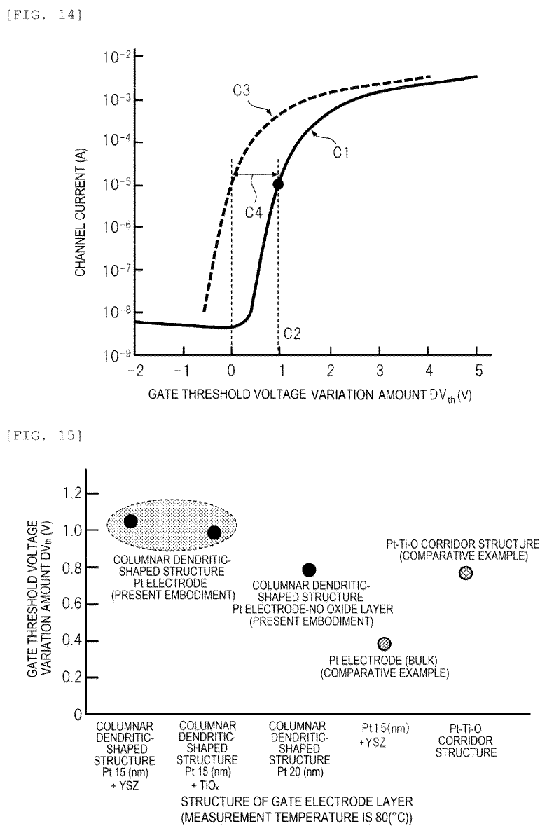

FIG. 14 is a diagram illustrating a threshold voltage variation amount DVₜₕ. In FIG. 14, a graph showing the relationship between the gate threshold voltage of the FET and a channel current is drawn. In the FET having a current-voltage characteristic C1 as shown in FIG. 14, a steady state, that is, the gate threshold voltage before gas detection is C2 (here, the gate voltage at 10⁻⁵ A), and when a current-voltage characteristic shifts due to gas adsorption and becomes C3, a voltage difference C4 before and after gas detection is obtained.

FIG. 15 is a diagram showing a relationship between the structure of the gate electrode layer and the gate threshold voltage variation amount DVₜₕ. The gate threshold voltage variation amount in the relationship shown in FIG. 15 is a value measured under a condition that a measurement temperature of the gate electrode layer is 80° C.

As shown in FIG. 15, when the structure of the gate electrode layer is a Pt—Ti—O corridor structure, the gate threshold voltage variation amount DVₜₕ is about 0.8 V. When the structure of the gate electrode layer is a structure in which a Pt electrode layer (bulk) having a film thickness of 15 nm is laminated on a YSZ film, the gate threshold voltage variation amount DVₜₕ is about 0.4 V.

Regarding this, when the gate electrode layer has a structure in which a Pt catalytic metal layer having a film columnar dendritic-shaped structure and a film thickness of 15 nm is laminated on the YSZ film, the gate threshold voltage variation amount DVₜₕ is about 1.05 V. When the gate electrode layer has the structure in which the Pt catalytic metal layer structure having the columnar dendritic-shaped structure and the film thickness of 15 nm is laminated on a TiOₓ film, the gate threshold voltage variation amount DVₜₕ is about 0.95 V. Further, when the gate electrode layer has a structure that includes only a catalytic metal layer having a columnar dendritic-shaped structure and does not include an oxide layer, the gate threshold voltage variation amount DVₜₕ is about 0.8 V.

From these results, it can be seen that when the gate electrode layer has the structure according to the present embodiment, the using amount of Pt is about half that of the Pt—Ti—O corridor structure, but the threshold voltage variation amount DVₜₕ is equal to or greater than that of the Pt—Ti—O corridor structure. This shows that the specific surface area is dramatically increased with respect to the using amount of catalytic metal because the catalytic metal layer has a columnar dendritic-shaped structure, making it possible to perform highly sensitive gas detection with a small using amount of catalytic metal.

As described above, according to the present embodiment, the catalytic metal layer having the columnar dendritic-shaped structure is used for the gate electrode layer of the gas sensor FET, and thus, the specific surface area of the catalytic metal layer is increased, and the gas sensor FET with improved gas detection sensitivity can be implemented. In particular, a highly sensitive gas sensor FET suitable for detecting hydrogen gas or detecting impurity gas in hydrogen gas or nitrogen gas, and a method for manufacturing the same can be provided.

In addition, in an FET sensor having a corridor structure made of catalytic metal/oxide in the related art, a combination of materials that can be used to form the corridor structure is limited to Pt—TiOₓ, Pt—MoOₓ, and the like, and a specific surface area is also limited to a maximum of about 10 m²/g, making it difficult to detect gas more efficiently. In addition, materials that can be used as a catalytic metal layer to detect various kinds of gas are limited, and thus, it is difficult to expand material options for the catalytic metal layer.

On the other hand, according to the method for manufacturing a gas sensor FET according to the embodiment of the invention, the catalytic metal layer uses a porous catalytic metal that can be implemented by film formation only, regardless of a surface state. According to a film formation method such as sputtering, a film is formed under appropriate conditions under high pressure conditions, and in addition to a usual columnar structure in the z-axis direction, many fine structures are formed in horizontal and oblique directions. By using this catalytic metal layer as a detection film, efficient gas detection is possible. In addition, a lower oxide layer that is necessary in a sensor having a corridor structure and made of catalytic metal/oxide in the related art and the resulting variation in film properties are eliminated, and a using amount of a catalytic metal can be dramatically reduced.

According to the present embodiment, the catalytic metal layer having the columnar dendritic-shaped structure in which a plurality of columnar dendritic-shaped structures are arranged two-dimensionally is formed by high-pressure sputtering, and thus, it is possible to increase the specific surface area with respect to the using amount of the catalytic metal, and improve gas detection sensitivity while reducing material costs.

According to the present embodiment, since the adhesive layer is not necessarily required to be inserted between the gate insulating layer and the catalytic metal layer on the semiconductor substrate, it is possible to simplify the structure and reduce the number of steps during manufacturing.

In the gas sensor FET according to the present embodiment, the specific surface area of the catalytic metal layer having the columnar dendritic-shaped structure is preferably within a range of 20 m²/g to 150 m²/g. This is because, from the measurement results showing the relationship between the specific surface area of the catalytic metal layer and the variation amount of the gate threshold voltage, the variation amount of the gate threshold voltage, which is directly linked to the gas detection sensitivity, corresponds to a sufficiently high range, as compared with a case where the gate electrode has the corridor structure in the related art.

The sputtering pressure in a case of forming the catalytic metal layer is preferably 5 Pa or more. This is because, from the measurement results showing the

### Relationship Between the Sputtering Pressure and the

specific surface area of the catalytic metal layer, the condition of the sputtering pressure of 5 Pa or more is derived as the condition in which the specific surface area of the catalytic metal layer becomes 20 m2/g or more, which is preferably. Note that a preferable upper limit of the sputtering pressure cannot be uniquely determined, but 50 Pa or less may be a realistic guideline based on specifications of the sputtering system, the knowledge and experience of the inventors, or the like.

From previous research results of the inventors, it is assumed that the catalytic metal layer is made of, for example, platinum, nickel, or a mixed material of platinum and nickel, and these are practical examples.

From previous research results of the inventors, it is assumed that the catalytic metal layer is made of at least one metal of palladium, rhodium, gold, silver, ruthenium, iridium, and copper, or a mixed material in which platinum or nickel is added to the at least one metal, and these are practical examples.

From previous research results of the inventors, it is assumed that the metal oxide film is, for example, yttrium-zirconium oxide, gadolinium-cerium oxide, or titanium oxide, and these are practical examples.

From previous research results of the inventors, it is assumed that a thickness of the metal oxide film is, for example, 5 nm or more and 30 nm or less, and this range is a practical example.

The electrode structure having a dendritic-shaped structure, which is a characteristic of the invention, is not limited to the FET-type sensor, and can be applied to, for example, a capacitor-type sensor.

### Embodiment 2

A gas sensor including the gas sensor FET according to the embodiment of the invention, a reference FET for referencing the gate threshold voltage, and a heater, which are disposed on the same substrate, is also an embodiment of the invention. For example, the gas sensor FET, the heater, and the reference FET are disposed on the same substrate. The entire substrate is heated to a predetermined temperature range by the heater, so that variation characteristics in the gate threshold voltages of the gas sensor FET and the reference FET approach expected characteristics and become stable. Then, the gate threshold voltage of the reference FET and the gate threshold voltage of the gas sensor FET are monitored, and the gas detection is performed based on a comparison result of the monitored gate threshold voltages. According to such a gas sensor, it is possible to perform highly reliable gas detection that is not easily affected by changes in an environmental temperature.

The embodiments of the invention have been described above, but these embodiments and examples are merely aspects of carrying out the invention, and the invention is not limited to these aspects, and various modifications are possible within the scope of the invention.

### Reference Signs List

1 gas sensor FET
11 semiconductor substrate
12 n-type semiconductor
13 gate electrode layer
14 protective layer
132 gate insulating layer
133 oxide layer
134 catalytic metal layer
1341 dendritic-shaped structure
1341a columnar portion
1341b fine structure portion

1. A gas sensor FET comprising:
a semiconductor substrate; and
a gate electrode layer formed on the semiconductor substrate and functioning as a gas detection unit, wherein
the gate electrode layer has a catalytic metal layer including a side exposed to an atmosphere,
the side of the catalytic metal layer exposed to the atmosphere has a structure in which a plurality of dendritic-shaped structures, each including a plurality of fine structure portions smaller and finer than a columnar portion formed on a surface of the columnar portion, are two-dimensionally arranged, and
the plurality of dendritic-shaped structures are electrically connected to each other.

2. The gas sensor FET according to claim 1, wherein the fine structure portions have a leaf shape, a plate shape, or a grain shape.

3. The gas sensor FET according to claim 1, wherein a specific surface area of the catalytic metal layer-film is 20 m²/g or more to 150 m²/g or less.

4. The gas sensor FET according to claim 1, wherein
a thickness of the catalytic metal layer is 5 nm or more and 500 nm or less.

5. The gas sensor FET according to claim 1, wherein
the catalytic metal layer is made of platinum, nickel, or a mixed material of platinum and nickel.

6. The gas sensor FET according to claim 1, wherein
the catalytic metal layer is made of at least one metal of palladium, rhodium, gold, silver, ruthenium, iridium, and copper, or a mixed material in which platinum or nickel is added to the at least one metal.

7. The gas sensor FET according to claim 1, wherein
the gate electrode layer has an oxide layer that is in contact with the catalytic metal layer and is disposed on a side opposite to the side exposed to the atmosphere.

8. The gas sensor FET according to claim 7, wherein
the oxide layer is made of yttrium-zirconium oxide, gadolinium-cerium oxide, or titanium oxide.

9. The gas sensor FET according to claim 7, wherein
a thickness of the oxide layer is 5 nm or more and 30 nm or less.

10. A gas sensor comprising:
the gas sensor FET according to claim 1 disposed on a substrate;
a reference FET disposed on the substrate and configured to reference a gate threshold voltage; and
a heater disposed on the substrate and configured to heat the substrate to a predetermined temperature range.

11. A method for manufacturing a gas sensor FET including a semiconductor substrate and a gas detection unit that is formed on the semiconductor substrate, constitutes a gate electrode layer, and includes a catalytic metal layer having a side exposed to an atmosphere, the method comprising:
a catalytic metal layer forming step of forming the catalytic metal layer by sputtering at a sputtering pressure of 5 Pa or more.

12. The method for manufacturing a gas sensor FET according to claim 11, further comprising:
a surface treatment step of activating a catalytic effect by performing a surface treatment of oxidation or reduction on the formed catalytic metal layer after the catalytic metal layer forming step.

13. The method for manufacturing a gas sensor FET according to claim 12, wherein
after a part of a protective layer formed on the semiconductor substrate is removed, a gate insulating layer is formed on a portion of the semiconductor substrate where the protective layer is removed, and the catalytic metal layer is formed on the formed gate insulating layer.

14. The method for manufacturing a gas sensor FET according to claim 13, further comprising:
a wiring material connection step of connecting a wiring material to the catalytic metal layer, wherein
a material having oxidation resistance is used as the wiring material.
